SEQUENCE LISTING (41 sequences)

SEQ ID NO: 1 - DNA, 32 bp, Escherichia coli
atatatggatccgtatcggggaccaaaatggc

SEQ ID NO: 2 - DNA, 32 bp, Escherichia coli
aaatttccatggaacttccaggcccgccatag

SEQ ID NO: 3 - DNA, 22 bp, Escherichia coli
caattgtgagcgctcacaattt

SEQ ID NO: 4 - DNA, 30 bp, Escherichia coli
ctagaaattgtgagcgctcacaattgggcc

SEQ ID NO: 5 - DNA, 76 bp, Escherichia coli
ctagaatatcgaattgtgagcgctcacaattctattccccgggaattgtgagcgctcacaattgtatctaggccta

SEQ ID NO: 6 - DNA, 76 bp, Escherichia coli
ctagtaggcctagatacaattgtgagcgctcacaattcccggggaatagaattgtgagcgctcacaattcgatatt

SEQ ID NO: 7 - DNA, 36 bp, Escherichia coli
aattttctcgagtagggataacagggtaatggtacc

SEQ ID NO: 8 - DNA, 32 bp, Escherichia coli
ctagttacgcgtacatgtcagatcctcttcgg

SEQ ID NO: 9 - DNA, 68 bp, Escherichia coli
ggccgcctcgagaatttgtattttcagggtgatctccgtggatctattaccctgttatccctagagct

SEQ ID NO: 10 - DNA, 60 bp, Escherichia coli
ctagggataacagggtaatagatccacggagatcaccctgaaaatacaaattctcgaggc

SEQ ID NO: 11 - DNA, 53 bp, Escherichia coli
aatgggctgaagaccgttagactctaattgtgagcgctcacaattcaatcctc

SEQ ID NO: 12 - DNA, 51 bp, Escherichia coli
ctgaaaatacaaattctcgaggattgaattgtgagcgctcacaattagagt

SEQ ID NO: 13 - DNA, 20 bp, Escherichia coli
ctaacggtcttcagcccatt

SEQ ID NO: 14 - DNA, 39 bp, Escherichia coli
ccgaaggagacgccaccatggtgacttctaatgttgtcc

SEQ ID NO: 15 - DNA, 38 bp, Escherichia coli
ggccgctagtcgacgggatcctaacggtcttcagccca

SEQ ID NO: 16 - DNA, 20 bp, Escherichia coli
ttccaggggcccgaaggaga

SEQ ID NO: 17 - DNA, 20 bp, Escherichia coli
attctagtgcggccgctagt

SEQ ID NO: 18 - DNA, 20 bp, Escherichia coli
ggaagttctcttccaggggc

SEQ ID NO: 19 - DNA, 20 bp, Escherichia coli
agcgctcacaattctagtgc

SEQ ID NO: 20 - DNA, 20 bp, Escherichia coli
gtggaagtctggaagttctc

SEQ ID NO: 21 - DNA, 70 bp, Escherichia coli
taacagggtaatagatccacggagatcaccctgaaaatacaaattctcgactaacggtcttcagcccatt

SEQ ID NO: 22 - DNA, 36 bp, Escherichia coli
ccgaaggagacgccaccatggaggagccgcagtcag

SEQ ID NO: 23 - DNA, 38 bp, Escherichia coli
ggccgctagtcgacgggatctcagtctgagtcaggccc

SEQ ID NO: 24 - DNA, 36 bp, Escherichia coli
ccgaaggagacgccaccatgactgcggagctgcagc

SEQ ID NO: 25 - DNA, 20 bp, Escherichia coli
ggccgctagtcgacgggatc

SEQ ID NO: 26 - DNA, 20 bp, Escherichia coli
gcaaaacatcttgttgaggg

SEQ ID NO: 27 - DNA, 20 bp, Escherichia coli
gacttgcacgtactcccctg

SEQ ID NO: 28 - DNA, 20 bp, Escherichia coli
catgtagttgtagtggatgg

SEQ ID NO: 29 - DNA, 20 bp, Escherichia coli
ggttggctctgactgtacca

SEQ ID NO: 30 - DNA, 20 bp, Escherichia coli
gagaggagctggtgttgttg

SEQ ID NO: 31 - DNA, 20 bp, Escherichia coli
agcactaagcgagcactgcc

SEQ ID NO: 32 - DNA, 20 bp, Escherichia coli
tactcccctgccctcaacaa

SEQ ID NO: 33 - DNA, 20 bp, Escherichia coli
gactgtaccaccatccacta

SEQ ID NO: 34 - DNA, 20 bp, Escherichia coli
gagcactgcccaacaacacc

SEQ ID NO: 35 - DNA, 20 bp, Escherichia coli
ccctcaacaagatgttttgc

SEQ ID NO: 36 - DNA, 20 bp, Escherichia coli
ccatccactacaactacatg

SEQ ID NO: 37 - DNA, 20 bp, Escherichia coli
caacaacaccagctcctctc

SEQ ID NO: 38 - DNA, 51 bp, Escherichia coli
ttcttcaggttaacccaacagaaggctcgagaaggtatattgctgttgaca

SEQ ID NO: 39 - DNA, 19 bp, Escherichia coli
cctaggtaatacgactcac

SEQ ID NO: 40 - DNA, 20 bp, Escherichia coli
aaggtatattgctgttgaca

SEQ ID NO: 41 - DNA, 50 bp, Escherichia coli
gtaatccagaggttgattgttccagacgcgtcctaggtaatacgactcac

(19) United States
(12) Patent Application Publication
(10) Pub. No.: US 2007/0292954 A1
(43) Pub. Date: Dec. 20, 2007

(54) Generation of recombinant DNA by sequence-and ligation-independent cloning

(75) Inventor: Stephen Elledge, Brookline, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 11/785,599

(22) Filed: Apr. 19, 2007

Related U.S. Application Data
(60) Provisional application No. 60/794,185, filed on Apr. 21, 2006.

(51) Int. Cl. C12N 15/87; C12N 9/12

(52) U.S. Cl. 435/488; 435/194

(57) ABSTRACT

The present invention is directed methods for cloning DNA by homologous recombination. The methods can be used without a need for ligases or restriction enzymes and allow for the rapid alignment of multiple DNA fragments.

### CROSS REFERENCE TO RELATED APPLICATIONS

The present application claims priority to, and the benefit of, U.S. provisional application 60/794,185 filed on Apr. 21, 2006. This prior application is hereby incorporated by reference in its entirety.

### FIELD OF THE INVENTION

The present invention is in the field of recombinant DNA technology and is directed to methodology for cloning DNA by homologous recombination, without the need for ligases.

### BACKGROUND OF THE INVENTION

The assembly of recombinant DNA by restriction enzyme cutting and religation was a crowning achievement of biology in the 20^{th} century (Smith, et al., J. Mol. Biol. 51:379-391 (1970); Danna, et al., Proc. Natl. Acad. Sci. USA 68:2913-2917 (1971); Cohen, et al., Proc. Natl. Acad. Sci. USA 70:3240-3244 (1973); and Backman, et al., Cell 13:65-71 (1978)). Many variations on this theme have emerged that allow greater precision to be achieved with respect to sequence alterations and sites of junctions of recombinant molecules. Two methods that made critical improvements are site-directed mutagenesis (Hutchison, et al., J. Biol. Chem. 253:6551-6560 (1978)) and the polymerase chain reaction (PCR) (Rumsby, PCR Methods Mol. Biol. 324:75-89 (2006); Saiki, et al. Science 230:1350-1354 (1985)). Site-directed mutagenesis permits alteration of specific sequences to allow structure-function studies of molecules. PCR has made several contributions including the ability to select a precise sequence from low concentrations of DNA and to place specific sequences at fragment ends to allow conventional assembly with other fragments. PCR has also been used to introduce changes into gene sequences (Ho, et al., Gene 77:51-59 (1989)).

Today, the DNA sequence and coding capacities of whole organisms are being determined. This presents the opportunity to manipulate and analyze large sets of genes for genetic and biochemical properties. Furthermore, a new field, synthetic biology, is emerging which uses complex combinations of genetic elements to design circuits with novel properties. These new endeavors require the development of new cloning technologies. Three recombinational cloning methods have emerged for accomplishing parallel processing of large gene sets. Two utilize in vitro site-specific recombination, the Univector Plasmid-fusion System and Gateway (Liu, et al., Current Biology 8:1300-1309 (1998); Hartley, et al., Genome Res. 10:1788-1795 (2000); Walhout, et al. Methods Enzymol. 328:575-592 (2000); Bethke, et al., Nucleic Acids Res. 25:2828-2834 (1997); Nebert, et al., Ann. N.Y. Acad. Sci. 919-148-170 (2000); and Siegel, et al. Genome Res. 14:1119-1129 (2004)). The third, MAGIC, is an in vivo method that relies upon homologous recombination and bacterial mating (Li, et al., Nat. Gen. 37:311-319 (2005)).

These methods offer a uniform and seamless transfer of genes from one expression context to another, thereby allowing different clones to be treated identically. However they lack important features. First, they do not generally facilitate initial assembly of the gene of interest into the origin plasmid. The exception, Gateway, requires the use of expensive enzymes for initial cloning, and requires specific long sequences on each primer that contain recombination sites. Secondly, these methods are useful only for cloning into specific vectors containing defined sequences. If a cloning reaction requires a specialty assembly, i.e. replacing a fragment in an existing plasmid, perhaps within a gene, these methods cannot be employed. Finally, these methods generally allow only the combination of two fragments in a single experiment.

### SUMMARY OF THE INVENTION

Homologous recombination has important advantages over site-specific recombination in that it does not require specific sequences. Two types of homologous recombination exist in E. coli, RecA-mediated recombination and a RecA-independent pathway called single-strand annealing, SSA (Amundsen, et. al., Cell 112:741-744 (2003); Kuzminov, Microbiol. Mol. Biol. Rev. 63:751-813 (1999)). The present application addresses the limitations of current systems by the development of a new in vitro homologous recombination method called Sequence- and Ligation-Independent Cloning, SLIC. Homologous recombination intermediates, such as large gapped molecules assembled in vitro by RecA or single-strand annealing, efficiently transform E. coli, removing the sequence constraints inherent in other methods. This system circumvents many problems associated with conventional cloning methods, providing a multifaceted approach for the efficient generation of recombinant DNA.

In its first aspect, the invention is directed to a method of generating recombinant DNA by homologous recombination without the use of ligases. This is accomplished by amplifying one or more target DNA molecules by the polymerase chain reaction (PCR) using a forward primer and a reverse primer, each of which is typically 15-100 (and more typically 15-50) nucleotides long. The forward primer should terminate at one end in sequence A and the reverse primer should terminate at one end in a different sequence, sequence B, both of which should usually be 15-100 (and more typically 15-50) nucleotides in length. In the next step, a single stranded terminal region, typically 15-100 nucleotides long, is generated in the amplified DNA molecules using exonuclease digestion so that, at one end, they have a 5′ overhang corresponding, at least in part, to sequence A and, at the other end, a 5′ overhang corresponding, at least in part, to sequence B. These fragments are then annealed with a linearized vector that terminates at each end with a single stranded region (again typically 15-100 and more typically 15-50 nucleotides in length). One end should have a sequence, C, that is exactly complementary to sequence A, and the other end should have a sequence, D, that is exactly complementary to sequence B. Once annealing is complete, the final step in the process involves transforming a host cell (preferably E. coli) with the annealed complexes that have been formed. Enzymes in the host cell will then fill in any missing nucleotides and join the annealed DNA fragments together. If desired, this recombinant vector may now be recovered from the host. Although this system has been described in terms of 5′ overhangs, it should be noted that 3′ overhangs can also be used.

The annealing of DNA fragments to vector may be done either in the presence of RecA (at a concentration of about 0.1-0.5 ng/μl and preferably 0.2-0.4 ng/μl) or in the absence of Rec A, but at a higher concentration (at least 0.5 ng/μl and preferably 0.7-10.0 ng/μl or higher). The generation of single stranded regions in PCR amplified DNA and in the vector can be accomplished using one or more exonucleases such as: lambda nuclease; T7 nuclease; Exonuclease III; and T4 polymerase.

In another aspect, the invention is directed to a method of generating recombinant DNA by homologous recombination without the use of ligases, in which single stranded regions are created by performing incomplete PCR. This may be contrasted with ordinary PCR in that the extension step in the final cycle of denaturation, annealing and extension is omitted. Thus, the method involves first amplifying one or more target DNA molecules in the manner described above but in which the final step in the PCR procedure does not include the extension of annealed DNA fragments with the Taq DNA polymerase, only denaturation and renaturation, which results in incompletely extended DNA molecules annealing to produce dsDNA with 5′ overhangs suitable for annealing. The amplified fragments are then annealed with vector and used to transform a host cell, again, as described above.

These procedures will be especially useful in the cloning of multiple fragments at once. For example, one can combine 2-10 double stranded DNA fragments, each of size 40 bp to 3.1 kb or longer, e.g., 5 kb or 10 kb. Each fragment should be made to terminate at one end in a single stranded segment, either A or A′, ending in a 5′ terminal phosphate and, at the other end, by a single stranded segment, B or B′ also ending in a 5′ terminal phosphate. These segments should typically be 15-100 nucleotides long and, more typically 15-50 nucleotides long. It should also be noted that 3′ overhangs will also work in this embodiment. Each A segment, should consist of a sequence that is exactly complementary to at least one B sequence, with the exact sequences being chosen based upon the order in which the fragments should be arranged. The DNA fragments produced should be subsequently, or concurrently, annealed to a linearized vector terminating at one end in a single stranded region having a sequence C, that is exactly complementary to sequence A′. The other end of the vector should also terminate in a single stranded region but with a sequence, D, that is exactly complementary to sequence B′. These end regions should typically be 15-100 nucloetides long. As in the procedures described previously, the final step is to transform a host cell, preferably E. coli, with the annealed complexes. Once inside the host cell, any sequence gaps will be filled in and the annealed fragments will be ligated together. In a preferred embodiment, each A and A′ segment has a sequence that is unique with respect to one another, i.e., there is a unique single stranded sequence associated with each fragment. This will promote the formation of a single arrangement of fragments annealed to the vector. Annealing reactions may be carried out either in the presence or absence of RecA, with the preferred host cell being E. coli. The preferred method of generating single stranded end regions in fragment is through the use of an exonuclease such as: lambda nuclease; T7 nuclease; Exonuclease III; and T4 polymerase.

The invention also encompasses kits containing the various components needed to carry out the procedures described above. For example a kit may include at least one oligonucleotide (e.g., 15-300 nucleotides in length) that terminates at one end in sequence A (e.g., 15-100 nucleotides long) and a vector that is, or can be, linearized to contain an end sequence that is exactly complementary to sequence A (e.g., 15-100 long). The kit should not include a DNA ligase but may include additional oligonucleotides (e.g., having a terminal sequence complementary to the other end of the vector) or additional components that may be used in the process, e.g., RecA or exonucleases.

### BRIEF DESCRIPTION OF THE DRAWINGS

FIG. 1: In vitro recombination of MAGIC vectors mediated by RecA. The figure shows a schematic for the production of recombinant DNA through in vitro homologous recombination and single-strand annealing.

FIG. 2: The dependency on RecA can be overcome by increased DNA concentrations. One μg of linear vector pML385 and 1 μg of 40 bp homology Skp1 insert fragment are treated with 0.5 U of T4 DNA polymerase for 1 hour. The vector and inserts are then diluted and annealed with and without RecA in a 1:1 molar ratio at different concentrations.

FIG. 3: Incomplete PCR (iPCR) and mixed PCR can be used to prepare inserts for SLIC cloning without nuclease treatment. The figure shows a schematic illustrating the production of mixed PCR products. Two PCR reactions are prepared using primer pairs P1F-P1R and P2F-P2R. Primers P1F and P2R are longer than P1R and P2F and produce 5′ and 3′ overhangs respectively. The two PCR products are mixed and heated to 95° C. for 5 minutes to denature, and then cooled slowly to room temperature to reannealed.

FIG. 4: Multi-fragment assembly using SLIC. A) A schematic is shown illustrating the 3-way SLIC reaction with lacO oligos. B) A schematic is shown illustrating the 5-way SLIC reaction in which T4 DNA polymerase-treated linear vector, pML385, and inserts with different amounts of homology are annealed in equalmolar ratio and transformed.

### DETAILED DESCRIPTION OF THE INVENTION

Recombinant DNA Methodology

The present invention is based upon studies demonstrating an efficient method for cloning that does not require the use of ligases. The methodology can be applied to the cloning of a single known DNA sequence or to the transfer of a sequence from one vector to another. In these cases, PCR primers will be designed based upon known DNA sequences flanking the target sequence, i.e., the DNA to be cloned. Alternatively, the methodology can be used to clone entire libraries using random DNA primers. The procedure is especially useful in the rapid assembly of numerous DNA fragments into a specific ordered arrangement within a vector.

In carrying out the present methods, nuclease digestion may be used to expose single stranded complementary or substantially complementary sequences on or near the ends of nucleic acid molecules and vectors. The complementary or substantially complementary ends thus revealed are capable of being annealed. Complementary nucleotides are A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, and preferably 90%, 95%, or 100%. “Completely” or “exactly” complementary sequences have no mismatches at all, i.e., all A's on one strand are aligned with T's on the other, all G's with C's etc.

“Hybridization” refers to the process in which two single-stranded polynucleotides bind non-covalently to form a stable double-stranded polynucleotide. “Hybridization conditions” will typically include salt concentrations of less than about IM, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Generally, stringent conditions are selected to be about 5° C., lower than the Tm for the specific sequence at s defined ionic strength and pH.

In one example of the invention, recombinant DNA is formed by annealing nucleic acid fragments to vectors with complementary or substantially complementary ends such that an overhang region is created from an end of the vector. The overhang region may contain some sequences that are not complementary in addition to some that are complementary. The nucleic acid fragment may be prepared and treated with nucleases to create a nucleic acid fragment with ssDNA ends that are complementary or substantially complementary to nuclease-treated ends of the vector. The annealed complex may be transformed into a bacterial cell, such as E. coli. Such prepared nucleic acid fragments may exhibit increased efficiency in transformation. Alternatively, a nucleic acid fragment and a vector may be treated with a nuclease to create ssDNA ends that are capable of annealing such that an overhang region is excluded. In this example, the nucleic acid fragment is treated with any exonuclease, such as lambda nuclease, T7 nuclease, Exonuclease III, and/or the exonuclease function of T4 polymerase.

Nucleic acid molecule assemblies may be created by annealing multiple nucleic acid fragments with each other and/or multiple vectors. By way of example, 3-way, 5-way, or 10-way gene assemblies may be formed in which 3 nucleic acid molecules, 5 nucleic acid molecules, or 10 nucleic acid molecules are annealed together, respectively. Based on the nucleic acid fragments described, efficiency of annealing and/or transformation into cells may be enhanced. For example, creation of the multiple gene assemblies may be increased up to 80-fold or greater upon preparation of inserts and addition of the fragments. Also, in one example, an overhang region may be created, the overhang region being of any sequence. Hence, there is no particular required sequence in the overhang region.

RecA protein enhances the efficiency of formation of annealed complexes of nucleic acid fragments and/or vectors such that small amounts of nucleic acid molecules and/or vectors may be stimulated for production of recombinant molecules by up to 100-fold or higher. Enhanced production of recombinant molecules may be achieved in the presence of RecA even at low concentrations of nucleic acid molecules.

Any given fragment (generated, for example, by polymerase chain reaction or other methods) may be directionally subcloned and used for high throughput subcloning of open reading frames (ORFs) into a given vector. Also, ORFs may be linked to different promoters or selectable markers. In another example, site-directed mutagenesis of proteins may be accomplished in which any portion of a gene may be altered without the presence of restriction enzymes. Further, the gene may be reassembled with any sequence at any position. Also, fragments from one gene or coding sequence can be introduced into another gene, related gene, or coding sequence in frame.

Recombinant molecules can be assembled in vitro with any combination of fragments. The fragments may include, for example, coding sequences, non-coding sequences, gene regulatory elements, whole genes, markers (e.g. nutritional, drug-resistance, enzymatic, calorimetric, fluorescent, etc.), origins of replication, recombination sites, retroviral components, etc. Also, a kit may be provided for generating designed recombinant nucleic acid constructs. For example, the kit may contain a nuclease. Such a kit may additionally contain RecA.

Epitope Display Libraries and Identification of Disease Biomarkers

In an entirely different aspect, the invention is concerned with nucleic acid molecules that contain a coding sequence for all or part of a human protein. A plurality of nucleic acid molecules may be created for covering all or substantially all of the genes of the human genome. The plurality of coding segments (including, without limitation, short coding sequences) may be provided on a microarray, substrate or plurality of substrates (e.g., microtiter wells, beads, microparticles and the like) and may each further encode a corresponding peptide or protein. The encoded protein may further be utilized in protein or peptide display technologies.

In one example, each member of a plurality of short coding sequences may encode a corresponding peptide. Each of the encoded and/or expressed peptides may overlap in its amino acid sequence with at least one other synthesized peptide such that all or substantially all, of the peptides encoded by the human genome are covered by the library. Such coding sequences may encode antigenic peptide sequences, such as epitopes.

A biological sample from a subject, may be brought into contact with a set of peptides containing linear epitopes. For example, the peptides may be provided in a display library in which immunoprecipitation procedures are carried out with the biological sample from the subject. The biological sample may be, for example, patient serum or other bodily fluid from one or different individuals. Such a sample also may be a tissue or cell sample, or a lysate or homogenate thereof. A sample may be whole or fractionated; in some cases, a specific component (such as antibodies) may have been isolated from the sample for use in the methods of the invention. The sample may contain antibodies, including for example, autoantibodies, which, upon exposure to and/or incubation with a display library of peptide epitopes of the human genome, may bind to the peptide epitopes.

The epitopes thus captured may be identified in any variety of ways. For example, corresponding coding sequences may be amplified using PCR from the co-affinity purified nucleic acid sequences. The molecules thus obtained may further be hybridized to coding regions on a microarray containing a plurality of coding regions of the human genome. Alternatively, the coding sequences may be determined by such means without a pre-amplification step. Hence, a signature of auto-antibodies present in the patient sample may be determined.

“Microarray” refers to a type of multiplex assay product that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable.

Typically, the oligonucleotides or polynucleotides on microarrays are single stranded and are covalently attached to the solid phase support, usually by a 5′ end or a 3′-end. The density of non-overlapping regions containing nucleic acids in a microarray is typically greater than 100 per cm², and more preferably, greater than 1000 per cm². Microarray technology relating to nucleic acid probes is reviewed in the following exemplary references: Schena, editor, Microarrays: A Practical Approach (IRL Press, Oxford, 2000); Southern, Current Opin. Chem. Biol. 2:404-410 (1998); Nature Genetics Supplement, 21:1-60 (1999); U.S. Pat. Nos. 5,424,186; 5,445,934; and 5,744,305. Microarrays may be formed in a variety of ways, as disclosed in the following exemplary references: Brenner, et al, Nature Biotechnology 18:630-634 (2000); U.S. Pat. No. 6,133,043; U.S. Pat. No. 6,396,995; U.S. Pat. No. 6,544,732; and the like.

“Microarrays” or “arrays” can also refer to a heterogeneous pool of nucleic acid molecules that is distributed over a support matrix. The nucleic acids can be covalently or noncovalently attached to the support. Preferably, the nucleic acid molecules are spaced at a distance from one another sufficient to permit the identification of discrete features of the array. Nucleic acids on the array may be non-overlapping or partially overlapping. Methods of transferring a nucleic acid pool to support media is described in U.S. Pat. No. 6,432,360. Bead based methods useful in the present invention are disclosed in PCT US05/04373.

“Amplifying” includes the production of copies of a nucleic acid molecule of the array or a nucleic acid molecule bound to a bead via repeated rounds of primed enzymatic synthesis. “In situ” amplification indicates that the amplification takes place with the template nucleic acid molecule positioned on a support or a bead, rather than in solution. In situ amplification methods are described in U.S. Pat. No. 6,432,360.

“Support” can refer to a matrix upon which nucleic acid molecules of a nucleic acid array are placed. The support can be solid or semi-solid or a gel. “Semi-solid” refers to a compressible matrix with both a solid and a liquid component, wherein the liquid occupies pores, spaces or other interstices between the solid matrix elements. Semi-solid supports can be selected from polyacrylamide, cellulose, polyamide (nylon) and crossed linked agarose, dextran and polyethylene glycol.

“Randomly-patterned” or “random” refers to non-ordered, non-Cartesian distribution (in other words, not arranged at pre-determined points along the x- or y-axis of a grid or at defined “clock positions,” degrees or radii from the center of a radial pattern) of nucleic acid molecules over a support, that is not achieved through an intentional design (or program by which such design may be achieved) or by placement of individual nucleic acid features. Such a “randomly-patterned” or “random” array of nucleic acids may be achieved by dropping, spraying, plating or spreading a solution, emulsion, aerosol, vapor or dry preparation comprising a pool of nucleic acid molecules onto a support and allowing the nucleic acid molecules to settle onto the support without intervention in any manner to direct them to specific sites thereon. Arrays of the invention can be randomly patterned or random.

“Heterogeneous” refers to a population or collection of nucleic acid molecules that comprises a plurality of different sequences. According to one aspect, a heterogeneous pool of nucleic acid molecules results from a preparation of RNA or DNA from a cell which may be unfractionated or partially-fractionated.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g. 10,000 monomers, or more. Oligonucleotides comprising probes or primers usually have lengths in the range of from 12 to 100 nucleotides, and more usually, from 18 to 40 nucleotides. Oligonucleotides and polynucleotides may be natural or synthetic. Unless otherwise indicated, whenever an oligonucleotide is represented by a sequence of letters, such as “ATGC,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine. Usually oligonucleotides comprise the four natural deoxynucleotides; however, they may also comprise ribonucleosides or non-natural nucleotide analogs.

“Oligonucleotide tag” or “tag” means an oligonucleotide that is attached to a polynucleotide and is used to identify and/or track the polynucleotide in a reaction. Usually, a oligonucleotide tag is attached to the 3′- or 5′-end of a polynucleotide to form a linear conjugate, sometime referred to herein as a “tagged polynucleotide,” or equivalently, an “oligonucleotide tag-polynucleotide conjugate,” or “tag-polynucleotide conjugate.” The following references provide guidance for selecting sets of oligonucleotide tags appropriate for particular embodiments: U.S. Pat. No. 5,635,400; Brenner, et al, Proc. Nat'l Acad. Sci. USA, 97:1665-1670 (2600); Shoemaker, et al, Nature Genetics 14:450-456 (1996); Morris et al, European patent publication 0799897A1; U.S. Pat. No. 5,981,179; and the like. In different applications of the invention, oligonucleotide tags can each have a length within a range of from 4 to 36 nucleotides, or from 6 to 30 nucleotides, or from 8 to 20 nucleotides, respectively. A tag that is useful in the present invention to identify samples captured from a specific patient or other source is of sufficient length and complexity to distinguish it from sequences that identify other patients or sources of DNA being assayed in parallel.

As set forth above, identification of the captured epitopes and antibodies may be accomplished in any variety of ways. In one embodiment captured phage encoding epitopes of interest can be sequenced or identified by hybridization to microarrays. Once identified, particular epitopes may be identified in any of a number of additional ways as peptide reagents. In one embodiment, labeled epitopes are detected via a microarray of tag epitopes. In this embodiment, for each different antibody (e.g., from distinct patients, patient samples or other sources) there is a unique labeled epitope tag. That is, the pair consisting of (i) the sequence of the epitope tag and (ii) a label that generates detectable signal are uniquely associated with a particular locus. The nature of the label on an epitope tag can be based on a wide variety of physical or chemical properties including, but not limited to, light absorption, fluorescence, chemiluminescence, electrochemi-luminescence, mass, charge, and the like. The signals based on such properties can be generated directly or indirectly. For example, a label can be a fluorescent molecule covalently attached to an epitope tag that directly generates an optical signal. Alternatively, a label can comprise multiple components, such as a hapten-antibody complex, that, in turn, may include fluorescent dyes that generated optical signals, enzymes that generate products that produce optical signals, or the like. Preferably, the label on a tag is a fluorescent label that is directly or indirectly attached to a tag. In one aspect, such fluorescent label is a fluorescent dye or quantum dot selected from a group consisting of from 2 to 6 spectrally resolvable fluorescent dyes or quantum dots.

Attachment of fluorescent labels are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26:227-259 (1991); and the like. Particular methodologies applicable to the invention are disclosed in the following sample of references: Fung, et al, U.S. Pat. No. 4,757,141; U.S. Pat. No. 5,151,507; U.S. Pat. No. 5,091,519.

In one aspect, one or more fluorescent dyes are used as labels for labeled target sequences, e.g. as disclosed by U.S. Pat. No. 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like. Labeling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. Commercially available fluorescent nucleotide analogues readily incorporated into the labeling oligonucleotides include, for example, Cy3-dCTP, Cy3-dUTP, Cy5-dCTP, Cy5-dUTP (Amersham Biosciences, Piscataway, N.J., USA), fluorescein-12-dUTP, tetramethylrhodamine-6-dUTP, Texas Red™.-5-dUTP, Cascade Blue™.-7-dUTP, BODIPY™. FL-14-dUTP, BODIPY™.R-14-dUTP, BODIPY™. TR-14-dUTP, Rhodamine Green™.-5-dUTP, Oregon GreenR™. 488-5-dUTP, Texas Red™.-12-dUTP, BODIPY™. 630/650-14-dUTP, BODIPY™. 650/665-14-dUTP, Alexa Fluor™. 488-5-dUTP, Alexa Fluor™. 532-5-dUTP, Alexa Fluor™. 568-5-dUTP, Alexa Fluor™. 594-5-dUTP, Alexa Fluor™. 546-14-dUTP, fluorescein-12-UTP, tetramethylrhodamine-6-UTP, Texas Red™.-5-UTP, mCherry, Cascade Blue™.-7-UTP, BODIPY™. FL-14-UTP, BODIPY™. TMR-14-UTP, BODIPY™. TR-14-UTP, Rhodamine Green™.-5-UTP, Alexa Fluor™. 488-5-UTP, Alexa Fluor™. 546-14-UTP (Molecular Probes, Inc. Eugene, Oreg., USA).

Biotin, or a derivative thereof, may also be used as a label on a detection oligonucleotide, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into a detection oligonucleotide and subsequently coupled to an N-hydroxy succinimide (NHS) derivitized fluorescent dye, such as those listed supra. In general, any member of a conjugate pair may be incorporated into a detection oligonucleotide provided that a detectably labeled conjugate partner can be bound to permit detection. As used herein, the term antibody refers to an antibody molecule of any class, or any subfragment thereof, such as an Fab.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified.

Auto-antibodies may be detected and characterized in a patient's sera or other sample. They may be present in patient sera in any number of conditions including, but not limited to, scleroderma, arthritis, multiple sclerosis, lupus, etc. A biological sample, such as patient serum, may be obtained from the patient and exposed or incubated with epitopes as described. The auto-antibodies may be identified to determine a signature of auto-antibodies in the sera. Hence, more effective and rapid diagnosis may be accomplished for the patent with subsequent directed therapy.

Also, auto-immune responses may be identified in various cancers. In one example, a slow growing tumor may be undetectable in the early stages of the disease. However, if the tumor or cancer causes the production of auto-immunity, identification of auto-antibodies in a patient sample may provide clues or early diagnosis of the development of the cancer even before the cancer is advanced enough to diagnose using alternative diagnostic modalities. Hence, in one example of the present invention, more effective and earlier diagnosis of cancer may be accomplished by identifying the presence of auto-antibodies in a patient's sample.

The peptides may also be used to search for proteins other than antibodies. For example, a linear protein or peptide segment may bind to a given target protein in vitro. Thus, the linear protein or peptide segment may be detected in a sample via binding to a target protein. Also, the target protein may contain an epitope encoded in the human genome. Thus, the process may further be used to identify proteins.

The embodiments herein include any feature or combination of features disclosed herein either explicitly or any generalization thereof. While the invention has been described with respect to specific examples including presently preferred modes of carrying out the invention, those skilled in the art will appreciate that there are numerous variations and permutations of the above described systems and techniques.

### EXAMPLES

The present example describes a novel cloning method SLIC (Sequence and Ligation-Independent Cloning) that allows the assembly of multiple DNA fragments in a single reaction using in vitro homologous recombination and single-strand annealing. SLIC mimics in vivo homologous recombination by relying on exonuclease generation of single strand DNA (ssDNA) overhangs on insert and vector fragments and the assembly of these fragments by recombination in vitro. SLIC inserts can be prepared by incomplete PCR (iPCR) or mixed PCR. SLIC allows efficient and reproducible assembly of recombinant DNA with as many as 5 and 10 fragments simultaneously. SLIC circumvents the sequence requirements of traditional methods and is much more sensitive when combined with RecA to catalyze homologous recombination. This flexibility allows much greater versatility in the generation of recombinant DNA for the purposes of synthetic biology.

A. Materials and Methods

Plasmid Construction

We amplified the PheS Gly294 gene by PCR using primers MZL561 and MZL562, and cloned into NcoI-BamHI cleaved pMAGIC1 to create pML385. We made the Lenti vector pMIA10-PheS in several steps. First, we annealed a pair of oligonucleotides, lacOF and lacOR, containing one lacO site, and inserted it into ApaI-XbaI cleaved GINmirCm^{R} to create GINmirCm^{R}-lacO. Second, we annealed another pair of oligonucleotides, 2-lacOF and 2-lacOR, containing two lacO sites, and inserted them into XbaI cleaved GINmirCm^{R}-lacO to generate pML410. We amplified the PheS Gly294 gene by PCR using primers MZL590 and MZL591, and cloned into the MluI-XhoI cleaved pML410 to create pMIA10-PheS.

To remove the lacO site from pML385, we created pML403 by annealing a pair of oligonucleotides, MZL571 and MZL572, and cloning them into NotI-SacI cleaved pML385. We made the plasmid tmGIPZ-pheS by inserting the PheS Gly294 gene, amplified by PCR using primers MZL590 and MZL591, into the MluI-XhoI cleaved tmGIPZ.

A list of primers and templates for PCR inserts are given in Table 1 and the primer sequences are given in Table 2.

Protocol for SLIC Sub-Cloning Using T4 DNA Polymerase Treated Inserts with RecA
1. Digest 2 μg of vector with restriction enzymes. Gel purify the vector and isolate the DNA using QIAEX II gel extraction kit. Quantitate the vector.
2. Inserts are amplified using Taq DNA polymerase. A 100 μl PCR reaction was set up with 250 μM of each dNTP, 0.5 μM of each primer, and 2.5 U of Taq DNA polymerase (from Eppendorf). Cycle as following: 94° C. for 45 seconds; 30 cycles of 94° C. for 45 seconds, 54° C. for 45 seconds, and 72° C. for 1 minute; 72° C. for 10 minutes. Add 20 U of DpnI to 100 μl of PCR products after PCR, incubate at 37° C. for 1 hour (not necessary if going from a MAGIC vector to ColE1 origin). The PCR products are purified by QIAquick PCR purification column.
3. Quantitate the inserts. We typically have 20 bp homology between the vector and the inserts. Take 1 μg of the vector and 1 μg of the inserts treat separately with 0.5 U of T4 DNA polymerase in T4 buffer (NEB) plus BSA in a 20 μl reaction at room temperature for 30 minutes. Stop the reaction by adding 1/10 volume of 10 mM dCTP and leave on ice.
4. Set up a 10 μl annealing reaction using 1:1 insert to vector ratio with 3 ng or less of a 3.1 kb vector (0.0015 μmol), 1× ligation buffer (NEB), appropriate amount of insert, 20 ng of RecA protein (Epicentre Biotechnologies) and water. Incubate in 37° C. for 30 minutes. Leave on ice or store in −20° C.
5. Add 5 μl of the annealed mixture into 150 μl of BW23474 chemical competent cells, incubate on ice for 30 minutes, heat shock at 42° C. for 45 seconds, return to ice for 2 minutes, add 0.9 ml of SOC, recover at 37° C. for 1 hour.
6. Plate 100 μl onto plates containing the appropriate antibiotics; incubate at 37° C. overnight (Cl-Phe is used for vectors that have PheS-G294 between restriction enzyme sites. We have found that most background comes from uncut vector in our preps and therefore we can select against it with Cl-Phe. However, Cl-Phe is not an essential step and usually only has a 2-fold effect on background.)

Protocol for SLIC Sub-cloning using iPCR or mixed PCR products
1. Digest 2 μg of vector with restriction enzymes. Gel purify the vector and isolate the DNA using QIAEX II gel extraction kit. Quantitate the vector.
2. Inserts are amplified using Taq DNA polymerase. A 100 μl PCR reaction was set up with 250 μM of each DNTP, 0.5 μM of each primer, and 2.5 U of Taq DNA polymerase (from Eppendorf). Cycle as following: 94° C. for 45 seconds; 30 cycles of 94° C. for 45 seconds, 54° C. for 45 seconds, and 72° C. for 1 minute; 72° C. for 10 minutes. Add 20 U of DpnI to 100 μl of PCR products after PCR, incubate at 37° C. for 1 hour. The PCR products were purified by QIAquick PCR purification column. Quantitate PCR products.
3. For iPCR insert, the PCR product is heated to 95° C. for 5 minutes to denature, cooled slowly to room temperature in 1 hour to renature, dilute and proceed to annealing reaction. For mixed PCR inserts, the two PCR products are mixed in equal amounts and heated to 95° C. for 5 minutes to denature, cooled slowly to room temperature for 1 hour to renature, dilute and proceed to annealing reaction.
4. Take 10 μl of the vector treat with 0.5 U of T4 DNA polymerase in T4 buffer (NEB) plus BSA in a 20 μl reaction at room temperature for 30 minutes. Stop the reaction by adding 1/10 volume of 10 mM dCTP and leave on ice.
5. Set up a 10 μl annealing reaction using 1:1 or higher insert to vector ratio with 150 ng of a 3.1 kb vector (0.074 pmol), 1× ligation buffer, appropriate amount of insert, and water. Incubate at 37° C. for 30 minutes. Leave on ice or store at −20° C.
6. Add 5 μl of the annealed mixture into 150 μl of BW23474 chemical competent cells, incubate on ice for 30 minutes, heat shock at 42° C. for 45 seconds, return to ice for 2 minutes, add 0.9 ml of SOC, recover at 37° C. for 1 hour.
7. Plate 100 μl onto plates containing the appropriate antibiotics; incubate in 37° C. for over-night.

SLIC

We digested the vector pML385 with NcoI-BamHI and gel purified it using a QIAEX II gel extraction kit. We amplified inserts using Taq DNA polymerase. After PCR, we added 20 U of DpnI to the PCR reaction and incubated at 37° C. for 1 hour to digest the template. We purified the inserts by QIAquick PCR purification column. We treated 1 μg of the vector and 1 μg of the inserts separately with 0.5 U of T4 DNA polymerase in 20 μl reactions at room temperature for different times depending on homology length. The optimal treatment for 20 bp overlap is 30 minutes and for 40 bp is 60 minutes. Reactions were stopped by adding 1/10 volume of 10 mM dCTP. We routinely use 150 ng of the vector and appropriate amount of inserts in a 1:1 or 2:1 insert to vector molar ratio in a 10 μl annealing reaction with 1× ligation buffer and incubation at 37° C. for 30 minutes. We transformed 5 μl of the annealing mix into 150 μl of chemically competent cells and plated on Cl-Phe plates. In cases with low DNA concentrations, electro-competent cells were used.

SLIC with iPCR or Mixed PCR Products

iPCR products were generated under the same conditions as regular PCR and purified by a QIAquick PCR purification column. We denatured the purified iPCR product at 95° C. and renatured slowly to room temperature in one hour. We annealed the vector and inserts at a 1:1 molar ratio and transformed.

We generated the two mixed PCR products separately using primer pairs P1F-P1R and P2F-P2R. Primers P1F and P2F are overlapping on the 3′ end but PIF is longer and contains a 5′ 30 bp homology region to the vector, tmGIPZ-pheS. Primers P1R and P2R are overlapping on the 3′ end but P2R is longer and contains the second 30 bp homology region to the vector. After PCR, we purified the two PCR products by QIAquick PCR purification columns, mixed in equal amounts, denatured at 95° C. for 5 minutes and renatured slowly to room temperature in one hour. We annealed the vector and inserts at 1:6 molar ratio and transformed.

B. Results

In Vitro Homologous Recombination with and without RecA

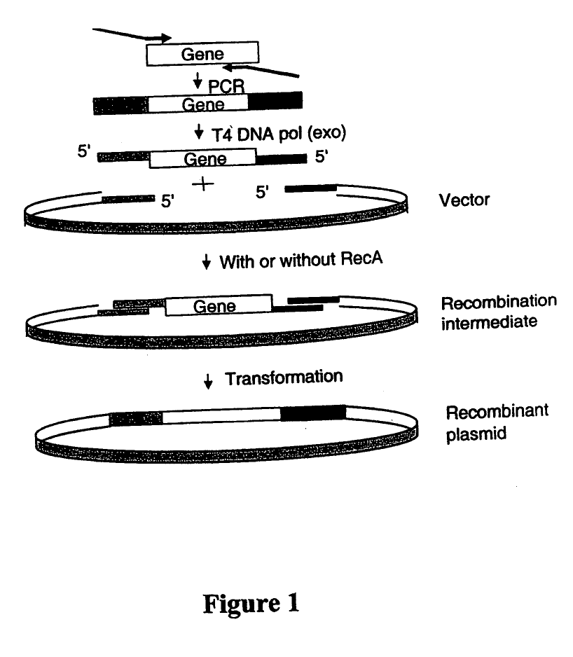

Homologous recombination in vivo depends upon a double-stranded break, generation of ssDNA by exonucleases, homology searching by recombinases, annealing of homologous stretches and repair of overhangs and gaps by enzymes that include resolvases, nucleases and polymerases. We reasoned it might be possible to generate recombination intermediates in vitro and introduce these into cells to allow the cells endogenous repair machinery to finish the repair to generate recombinant DNA (FIG. 1). To generate overhangs for homology searching, we employed exonucleases to chew back one strand to reveal ssDNA overhangs. We used both 3′ and 5′ exonucleases including T7 exonuclease, Lambda exonuclease, and T4 DNA polymerase. We chose T4 DNA polymerase, which produces 5′ overhangs, because it gave the best and most reproducible results and had the ability to terminate excision by addition of a single dNTP. We generated the vector by cleavage with a restriction enzyme and insert was generated by PCR. We treated with T4 DNA polymerase in the absence of dNTPs to generate overhangs, then incubated vector and insert with and without RecA protein and ATP to promote recombination, and transformed into E. coli. Vector alone gave some background we traced to a small amount of uncleaved vector. We reduced this in these experiments by placing the negative selectable marker pheS Gly294 between the restriction sites and plating on plates containing chloro-phenylalanine (Cl-Phe). However, in most cases Cl-Phe gives only a 2-fold reduction in background. Vector alone gave very few transformants, while incubation with equimolar insert fragments and RecA produced 400-600-fold stimulation over background. All 20 clones analyzed had the correct restriction map. We observed some stimulation over background without RecA, indicating that SSA could produce recombinants under these conditions. In the experiment shown in Table 3, 30 bp homology gave the greatest stimulation.

To examine how this method might apply to different vector and insert systems, we chose a vector/insert combination previously shown to efficiently recombine in vivo via MAGIC cloning. MAGIC donor vectors have greater than 60 bp homology with recipient vectors on each end and generate inserts by cleavage with I-SceI of both donor and recipient plasmids. The recipient used is a Lenti vector and the donor fragment is an shRNA cassette from an shRNA library. We incubated prepared fragments with and without RecA, electroporated into E. coli and selected for carbenicillin resistance. Without RecA, vector alone gave 1.3 transformants per ng of vector whereas vector plus insert gave 51. With RecA, vector alone gave 8 transformants per ng of vector whereas vector plus insert gave 3,900, a 500-fold stimulation of recombination, similar to what is seen in vivo. Ten of ten clones yielded restriction fragments consistent with the predicted restriction map. In addition, the isolation of 3,900 transformants per ng of vector means libraries can be transferred by this method in vitro without losing complexity.

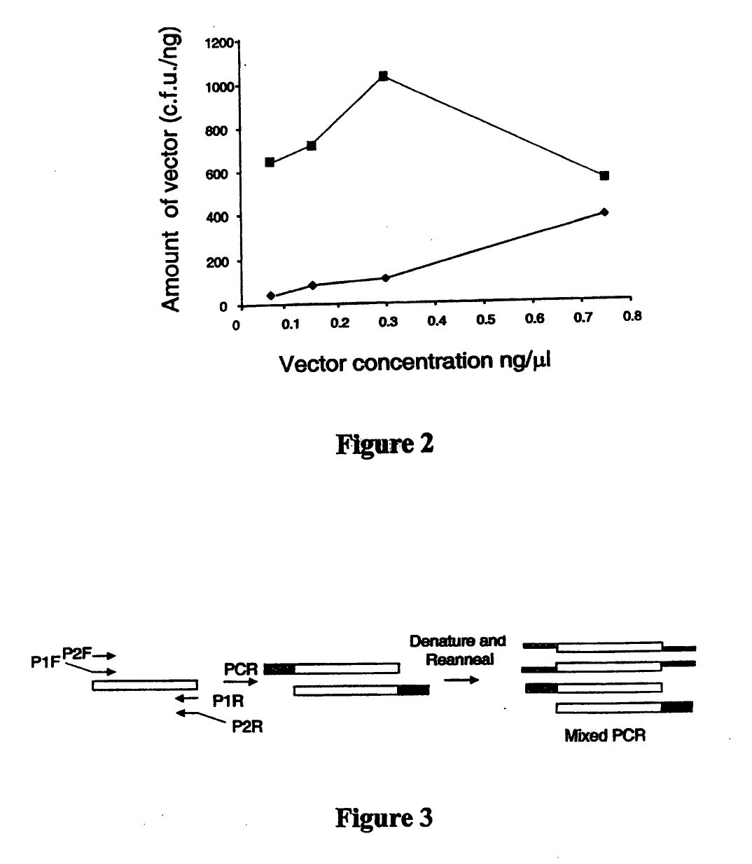

The requirement of RecA for efficient recombination suggested that at the DNA concentrations employed, efficient homology searching required enzymatic facilitation. We wondered if the less efficient SSA pathway could be made more efficient by increasing the concentration of input DNA to lessen dependency on RecA. Increasing both vector and insert by 10-fold greatly increased the efficiency of RecA-independent recombination (FIG. 2).

In Vitro Homologous Recombination Using iPCR or Mixed PCR Inserts

While optimizing the amount of T4 DNA polymerase for recombination, we observed that inserts derived from PCR displayed a low level of recombination without T4 treatment. One potential explanation is that incomplete synthesis of DNA during later cycles of PCR occurred such that some insert fragments might have 5′ overhangs. To test this, we used a fragment prepared by PCR and the identical fragment excised from a plasmid using restriction enzymes. Prior to T4 DNA polymerase treatment, the PCR-generated material gave 16-fold stimulation of transformation while the restriction fragment did not, confirming the incomplete PCR hypothesis (Table 4). We call this iPCR (incomplete PCR) and although inserts prepared by iPCR stimulate recombination with a lower efficiency it is a quick method for subcloning. We find that recombinant generation using iPCR generated inserts is more robust with higher insert concentration, likely because only a subset of iPCR molecules contain clonable overhangs. It should be noted if iPCR is used, one round of denaturation and renaturation without primer extension at the end should be performed prior to use in subcloning so molecules with overhangs on both ends will be present.

Mixing two PCR products, each of which has one homology region, upon denaturation and renaturation will yield 25% of resulting fragments with correct overhangs (FIG. 3). We tested this hypothesis using a large recipient plasmid vector, tmGIPZ-pheS (12 kb). At the higher insert to vector ratio of 6:1, mixed PCR inserts gave a 38-fold of stimulation over background, while a T4 DNA polymerase treated insert gave 70-fold stimulation (Table 4).

The ability to generate recombinant DNA by traditional methods varies depending upon the insert size and molar ratio with vector. The optimal insert to vector ratio varied between 2:1 and 4:1. By varying insert sizes we found that inserts up to 3.2 kb still showed robust homologous recombination in vitro. Plasmids where one fragment was as large as 7 kb assembled with good efficiency and one plasmid of 12 kb assembled at a reduced but sufficient efficiency, demonstrating that recombination with larger fragments is feasible.

In Vitro Homologous Recombination with Multiple Fragments

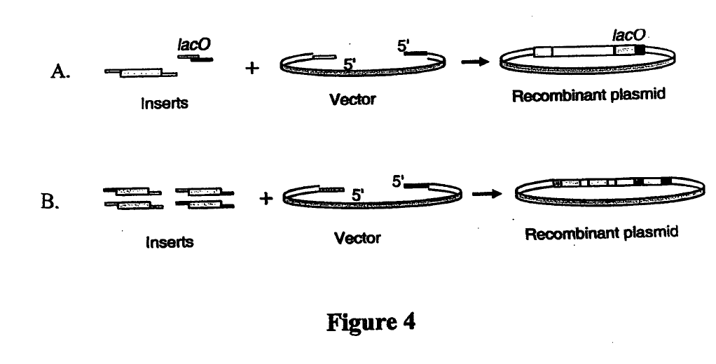

The efficiency of SLIC suggested it might be possible to generate recombinant DNA with multiple inserts. We first attempted a 3-way cloning consisting of a 0.6 kb insert and an approximately 75 bp lacO fragment generated by annealing two oligonucleotides that left 5′ overhangs on each side, one homologous to the 5′ end of the insert and the other to the vector (FIG. 4, panel A). Only the insert and vector were treated with T4 DNA polymerase. LacO was chosen because we had previously developed a genetic selection for subcloning lac operators in high copy which titrates out the endogenous lac repressor and induces a lac promoter driving bla. We reasoned if 3-way cloning was inefficient, rare recombinants could be selected. We plated recombinants on plates containing kanamycin to select for the vector, plus Cl-Phe to select against uncut vector. To determine the effects of selecting for lacO, we plated cells on these plates plus and minus carbenicillin. In the presence of carbenicillin, the background of vector and lacO alone upon transformation was extremely low and the presence of the third fragment stimulated recombinant formation by 20,000-fold (Table 5). To our surprise, in the absence of lacO selection, background increased to only 150 colonies/μg, but nearly 42,000 transformants and 280-fold stimulation were observed when all three fragments were present. Thus, 3-way assembly is robust.

To attempt a 5-way reaction, we generated four inserts of sizes from 275 bp to 400 bp by PCR (FIG. 4, panel B). The amount of overlapping homology was varied from 20 to 40 bp overlaps. There is an inherent problem with multiple fragment assembly because if assemblies occur on both ends of a vector, they may inhibit circularization if more than four fragments anneal to the vector ends. To minimize this potential problem, we did not use the excess of inserts that is optimal for 2-way assemblies. Inserts gave stimulation to each overhang class (Table 6). For 20 bp overhangs, a 14-fold stimulation was observed which rose to 20-fold with 30 bp overlap and 60-fold with 40 bp overlaps. Restriction analysis of the 40 bp recombinants revealed 20 of 20 had the correct restriction digest pattern. We chose 10 for complete sequence analysis. Eight had completely wild type sequence. One had a mutation in one of the primers and another had a PCR-induced mutation in one of the fragments.

We next attempted a 10-fragment assembly with 9 PCR-generated fragments of sizes ranging between 275 and 980 bp fragments with 40 bp overlaps inserted into a 3.1 kb vector. Unlike other assemblies, there was no stimulation of transformation upon addition of inserts. Restriction analysis revealed that 7 of 42 transformants had the correct restriction pattern. Unlike 5-way reactions, in this case clones were observed that had a subset of the insert fragments present due to faulty recombination. Nevertheless, nearly 20% of the 10-way assemblies were correct which is sufficient for complex component assembly in vitro. Similar results were obtained when reactions were performed with RecA. These data together indicate that the assembly of complex recombinant DNA assemblies can be achieved using in vitro homologous recombination.

C. Discussion

Unlike conventional methods that utilize restriction enzymes or site-specific recombinases, recombinant DNA assembled by SLIC achieves a seamless transfer of genetic elements in vitro without the need for specific sequences required for ligation or site-specific recombination. This is accomplished by harnessing the power of homologous recombination in vitro to assemble recombinant DNA that resemble recombination intermediates such as gapped or branched molecules which upon introduction into bacteria are repaired to regenerate a double-stranded, covalently closed plasmids.

Using bacterial recombinases such as RecA, recombinant DNA can be assembled efficiently with very small amounts of DNA. Homologous recombination events that occur in vivo such as those carried out by MAGIC cloning can be efficiently recapitulated in vitro using SLIC. This method can be used to assemble DNA made by PCR or restriction fragments. The only requirement is that the fragments to be assembled contain on their ends sequences of 20 bp or longer to allow stable annealing. Excision by the proofreading exonuclease of T4 DNA polymerase has proven to be the most reproducible and easiest to manipulate method for generating 5′ overhangs. Although much less efficient, iPCR also gives substantial stimulation of transformation. This might be sufficient for routine subcloning purposes although there is likely to be more variable depending on the completeness of the PCR synthesis.

The SLIC reactions described here that do not use RecA bear a resemblance to ligation independent cloning, LIC (Aslanidis, et al., Nuc. Ac. Res. 18:6069-6074 (1990); Haun, et al., Biotechniques 13:515-518 (1992); Aslanidis, et al., PCR Methods Appl. 4:172-177 (1994)). However, there are important differences. For LIC, PCR primers for inserts are designed to contain appropriate 5′ extension sequences lacking a particular dNTP that, after treatment with T4 DNA polymerase in the presence of the particular dNTP, generates specific 12 nucleotide ssDNA overhangs that are complementary to overhangs engineered into the vector. Importantly these overhangs have sequence constraints as they must be devoid of a common dNTP, which limits their use to specialized vectors bearing that sequence. The realization that alternative recombination intermediates with imprecise junctions such as large gaps and overhangs can be efficiently repaired in vivo completely liberates SLIC from the sequence constraints that the LIC method suffers. Having the ability to generate overlaps of greater lengths of unrestrained sequence provides much greater utility for SLIC and its combination with RecA makes it able to function at much lower DNA concentrations.

A significant advantage of the SLIC method is its flexibility with respect to sequence junctions. We have also shown that fragments with significant non-homologies of up to 20 nt at the ends can be assembled as long as the homologous regions are made single-stranded. Presumably these branched molecules are efficiently trimmed in vivo to generate recombinant plasmids. Unlike the site-specific recombination or restriction enzyme methods, SLIC allows alterations of fragments internal to a gene borne on a plasmid. For example it would be simple to introduce a PCR fragment into a restriction site in vitro even if that fragment contained multiple sites for the enzyme. Also, since the homologous junctions of fragments can be controlled, SLIC offers a new approach to the generation of site-directed mutations.

Among the strongest advantages offered by homologous recombination in vitro is the ability to assemble multimeric fragments. In conventional cloning experiments usually two and sometimes three fragments are assembled in one reaction assuming proper restriction sites are available. Our data indicate that five fragments can be easily assembled with high efficiency using SLIC and 10 fragments can be joined with reduced efficiency. The fidelity of the assembled molecules is limited only by the fidelity of PCR and the oligonucleotide primers used to generate the insert fragments. SLIC compares favorably with other multi-fragment cloning strategies such as multisite Gateway because SLIC allows complete control over junction fragments unlike Gateway which requires a defined site-specific recombination site between each fragment. Furthermore, SLIC works with PCR fragments while multi-site Gateway has only been demonstrated with cloned fragments on donor plasmids. The ability to assemble complex combinations of DNA sequence elements in defined orders will be particularly important in the field of synthetic biology. No attempts were made to optimize the 10-way assemblies and it is likely one could significantly improve the yield in future experiments. Thus, it is likely that molecules with greater than 10 fragments will be able to be assembled in the future.

The utility of the SLIC system is not limited to gene assembly. Genetic elements of any kind can be assembled using this system. One can now envision vectors being assembled in a combinatorial fashion from component parts. For example, using the highly efficient 5-way assembly one could combine an open reading frame together with a particular epitope tag, a tissue specific promoter, a retroviral vector together with a selectable marker of choice to generate a custom expression assembly. Thus, in the future vectors might exist in virtual form and be assembled in final form as needed. The advent of SLIC now brings the ability to manipulate DNA sequences with much greater facility than previously possible. Other complex assemblies such as homologous recombination targeting vectors could be assembled in one step by SLIC. These advances should save investigators significant amounts of time, effort and expense.

TABLE 1
PCR templates and primers

| PCR products | Templates | Primers |
|---|---|---|
| 0.6 kb Skp1 20 bp homology | pUNI20- | SkpNco20, SkpBam20 |
| | Skp1 | |
| 0.6 kb Skp1 30 bp homology | pMAGIC2- | TcNco30, TcBam30 |
| | Skp1 | |
| 0.6 kb Skp1 40 bp homology | pMAGIC2- | TcNco40, TcBam40 |
| | Skp1 | |
| 0.6 kb Skp1 50 bp homology | pMAGIC2- | SkpNco50, SkpBam50 |
| | Skp1 | |
| 1.2 kb hp53 20 bp homology | hp53 | hp53Nco20, |
| | | hp53Bam20 |
| 3.2 kb Usp28 20 bp homology | Usp28 | Usp28Nco20, |
| | | Usp28Bam20 |
| 0.6 kb Skp1 3-way lacO | pUNI20- | SkpNco20, MZL577 |
| | Skp1 | |
| 1.2 kb hp53 5-way PCR products | hp53 | hp53Nco20, p53-4w1; |
| (40 bp homology) | | p53-4w2, p53-20r; |
| | | p53-40f, p53-5w1; |
| | | p53-5w2, hp53Bam20 |
| 1.2 kb hp53 5-way PCR products | hp53 | hp53Nco20, p53-4w1; |
| (30 bp homology) | | p53-4w2-30, p53-20r; |
| | | p53-30f, p53-5w1; |
| | | p53-5w2-30, |
| | | hp53Bam20 |
| 1.2 kb hp53 5-way PCR products | hp53 | hp53Nco20, p53-4w1; |
| (20 bp homology) | | p53-4w2-20, p53-20r; |
| | | p53-20f, p53-5w1; |
| | | p53-5w2-20, |
| | | hp53Bam20 |
| 0.4 kb ShRNA cassette PCR | pSM2- | P1F, P2R; |
| products (30 bp homology) | ShRNA | P1F, P1R; |
| | | P2F, P2R |

TABLE 2
Primer sequences used in this study

| | |
|---|---|
| Primer name | Primer sequences |

| | |
|---|---|
| MZL561 | ATATATGGATCCGTATCGGGGACCAAAATGGC |
| | (SEQ ID NO:1) |
| MZL562 | AAATTTCCATGGAACTTCCAGGCCCGCCATAG |
| | (SEQ ID NO:2) |
| LacOF | CAATTGTGAGCGCTCACAATTT |
| | (SEQ ID NO:3) |
| LacOR | CTAGAAATTGTGAGCGCTCACAATTGGGCC |
| | (SEQ ID NO:4) |
| 2-lacOF | CTAGAATATCGAATTGTGAGCGCTCACAATTCTATTCCC |
| | CGGGAATTGTGAGCGCTCACAATTGTATCTAGGCCTA |
| | (SEQ ID NO:5) |
| 2-lacOR | CTAGTAGGCCTAGATACAATTGTGAGCGCTCACAATTCC |
| | CGGGGAATAGAATTGTGAGCGCTCACAATTCGATATT |
| | (SEQ ID NO:6) |
| MZL590 | AATTTTCTCGAGTAGGGATAACAGGGTAATGGTACC |
| | (SEQ ID NO:7) |
| MZL591 | CTAGTTACGCGTACATGTCAGATCCTCTTCGG |
| | (SEQ ID NO:8) |
| MZL571 | GGCCGCCTCGAGAATTTGTATTTTCAGGGTGATCTCCGT |
| | GGATCTATTACCCTGTTATCCCTAGAGCT |
| | (SEQ ID NO:9) |
| MZL572 | CTAGGGATAACAGGGTAATAGATCCACGGAGATCACCCT |
| | GAAAATACAAATTCTCGAGGC |
| | (SEQ ID NO:10) |
| MZL573 | AATGGGCTGAAGACCGTTAGACTCTAATTGTGAGCGCTC |
| | ACAATTCAATCCTC |
| | (SEQ ID NO:11) |
| MZL574 | CTGAAAATACAAATTCTCGAGGATTGAATTGTGAGCGCT |
| | CACAATTAGAGT |
| | (SEQ ID NO:12) |
| MZL577 | CTAACGGTCTTCAGCCCATT |
| | (SEQ ID NO:13) |
| SkpNco20 | CCGAAGGAGACGCCACCATGGTGACTTCTAATGTTGTCC |
| | (SEQ ID NO:14) |
| SkpBam20 | GGCCGCTAGTCGACGGGATCCTAACGGTCTTCAGCCCA |
| | (SEQ ID NO:15) |
| TcNco30 | TTCCAGGGGCCCGAAGGAGA |
| | (SEQ ID NO:16) |
| TcBam30 | ATTCTAGTGCGGCCGCTAGT |
| | (SEQ ID NO:17) |
| TcNco40 | GGAAGTTCTCTTCCAGGGGC |
| | (SEQ ID NO:18) |
| TcBam40 | AGCGCTCACAATTCTAGTGC |
| | (SEQ ID NO:19) |
| SkpNco50 | GTGGAAGTCTGGAAGTTCTC |
| | (SEQ ID NO:20) |
| SkpBam50 | TAACAGGGTAATAGATCCACGGAGATCACCCTGAAAATA |
| | CAAATTCTCGACTAACGGTCTTCAGCCCATT |
| | (SEQ ID NO:21) |
| hp53Nco20 | CCGAAGGAGACGCCACCATGGAGGAGCCGCAGTCAG |
| | (SEQ ID NO:22) |
| hp53Bam20 | GGCCGCTAGTCGACGGGATCTCAGTCTGAGTCAGGCCC |
| | (SEQ ID NO:23) |
| Usp28Nco20 | CCGAAGGAGACGCCACCATGACTGCGGAGCTGCAGC |
| | (SEQ ID NO:24) |
| Usp28Bam20 | GGCCGCTAGTCGACGGGATC |
| | (SEQ ID NO:25) |
| p53-4w1 | GCAAAACATCTTGTTGAGGG |
| | (SEQ ID NO:26) |
| p53-4w2 | GACTTGCACGTACTCCCCTG |
| | (SEQ ID NO:27) |
| p53-20r | CATGTAGTTGTAGTGGATGG |
| | (SEQ ID NO:28) |
| p53-40f | GGTTGGCTCTGACTGTACCA |
| | (SEQ ID NO:29) |
| p53-5w1 | GAGAGGAGCTGGTGTTGTTG |
| | (SEQ ID NO:30) |
| p53-5w2 | AGCACTAAGCGAGCACTGCC |
| | (SEQ ID NO:31) |
| p53-4w2-30 | TACTCCCCTGCCCTCAACAA |
| | (SEQ ID NO:32) |
| p53-30f | GACTGTACCACCATCCACTA |
| | (SEQ ID NO:33) |
| p53-5w2-30 | GAGCACTGCCCAACAACACC |
| | (SEQ ID NO:34) |
| p53-4w2-20 | CCCTCAACAAGATGTTTTGC |
| | (SEQ ID NO:35) |
| p53-20f | CCATCCACTACAACTACATG |
| | (SEQ ID NO:36) |
| p53-5w2-20 | CAACAACACCAGCTCCTCTC |
| | (SEQ ID NO:37) |
| P1F | TTCTTCAGGTTAACCCAACAGAAGGCTCGAGAAGGTATA |
| | TTGCTGTTGACA |
| | (SEQ ID NO:38) |
| P1R | CCTAGGTAATACGACTCAC |
| | (SEQ ID NO:39) |
| P2F | AAGGTATATTGCTGTTGACA |
| | (SEQ ID NO:40) |
| P2R | GTAATCCAGAGGTTGATTGTTCCAGACGCGTCCTAGGTA |
| | ATACGACTCAC |
| | (SEQ ID NO:41) |

TABLE 3
Recombinant stimulation by RecA.

| | Homology | | | |
|---|---|---|---|---|
| | 20 bp | 30 bp | 40 bp | 50 bp |
| Fragment | CFU/ng^{a} | CFU/ng^{a} | CFU/ng^{a} | CFU/ng^{a} |
| Vector^{b} only | <2.7 | 8.1 | 2.7 | <2.7 |
| Vector^{b} + Skp1 | 210 | 440 | 89 | 120 |
| Vector^{b} + RecA | 2.7 | <2.7 | <2.7 | <2.7 |
| Vector^{b} + Skp1 + RecA | 1,700 | 4,900 | 1,200 | 1,300 |

^{a}Colony forming units per ng of vector.
^{b}The recipient vector pML385 was linearized with Ncol-BamHI while the Skp1 fragment was prepared by PCR. Both were treated with T4 DNA polymerase to generate 5′ overhangs. This experiment was performed at low DNA concentration (0.075 ng/μl).

TABLE 4
Comparison between iPCR, mixed PCR and restriction
enzyme generated inserts on cloning efficiency.

| | No treatment | | +T4 treatment | |
|---|---|---|---|---|
| | CFU/ | Fold | CFU/ | Fold |
| Fragment | ng^{a} | induction | ng^{a} | induction |
| Vector 1^{b} only (3.1 kb) | 0.8 | 1 | 0.8 | 1 |
| Vector 1^{b} + iPCR fragment | 13 | 16 | 220 | 280 |
| Vector 1^{b} + restriction fragment | 1 | 1.3 | 650 | 810 |
| Vector 2^{c} only (12 kb) | 0.2 | 1 | 0.2 | 1 |
| Vector 2^{c} + mixed PCR fragment | 7.6 | 38 | — | — |
| Vector 2^{c} + T4 treated PCR | — | — | 14 | 70 |
| fragment | | | | |
| ^{a}Colony forming units per ng of vector. | | | | |
| ^{b}The linear vector 1 (pML385, 3.1 kb) was treated with T4 DNA polymerase. The 20 bp homology Skp1 insert generated by iPCR or the identical insert generated by Smal digestion were heated to 95° C. for 5 minutes to denature, and then cooled slowly to room temperature to re-anneal. For the +T4 lanes, inserts (iPCR and restriction fragment) were treated with T4 DNA polymerase. The vector and the appropriate | | | | |
| # amount of inserts (1:1 molar ratio) were then annealed and transformed. | | | | |

^{c}The linear vector2 (ptmGIPZ-pheS, 12 kb) and insert (using primer pair P1F-P2R) were treated with T4 DNA polymerase. The vector and the insert generated by T4 DNA polymerase were annealed at 1:2 molar ratio of vector to insert and transformed. The vector and the insert generated by mixed PCR were annealed at 1:6 molar ratio of vector to insert and transformed.

TABLE 5
Three-way SLIC with lacO selection.

| | Cl-Phe Kan Cb | Cl-Phe Kan |
|---|---|---|
| | CFU/μg^{a} | CFU/μg^{a} |
| Vector^{b} + lacO | 1.6 | 150 |
| Vector^{b} + insert + lacO | 36,000 | 42,000 |

^{a}Colony forming units per μg of vector.
^{b}T4 DNA polymerase treated pML403 and a 20 bp homology Skp1 insert were annealed with a pair of lacO oligos (1:1:1 molar ratio) and transformed.

TABLE 6
Five-way SLIC with different amounts of homology.

| | Homology | | |
|---|---|---|---|
| | 20 bp | 30 bp | 40 bp |
| Fragment | CFU/μg^{a} | CFU/μg^{a} | CFU/μg^{a} |
| Vector^{b} only | 390 | 410 | 360 |
| Vector^{b} + inserts | 5,300 | 8,700 | 22,000 |

^{a}Colony forming units per μg of vector.
^{b}T4 DNA polymerase treated pML385 and inserts with different amounts of homology were annealed in equimolar ratio and transformed.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method of generating recombinant DNA by homologous recombination without the use of ligases, comprising:
a) amplifying one or more target DNA molecules by the polymerase chain reaction (PCR) using a forward primer and a reverse primer, wherein
i) said forward primer terminates at its 5′ end in sequence A, wherein sequence A is 15-100 nucleotides in length;
ii) said reverse primer terminates at its 3′ end in sequence B, wherein sequence is 15-100 nucleotides long;
b) generating a single stranded terminal region 15-100 length in the amplified DNA molecules of step a);
c) annealing DNA fragments produced in step b) with a linearized vector, wherein one end of said vector terminates in a single stranded region having a sequence C that is exactly complementary to sequence A, and the other end of said vector terminates in a single stranded region having a sequence D that is exactly complementary to sequence B;
d) transforming a host cell with the annealed complexes formed in step c).

2. The method of claim 1, wherein said host cell is a bacterium.

3. The method of claim 2, wherein said bacterium is of the species E. coli.

4. The method of claim 1, wherein the annealing of step c) is carried out in the presence of RecA.

5. The method of claim 1, wherein the single stranded terminal regions of step b) are generated by digestion of said amplified DNA molecules using an exonuclease selected from the group consisting of: lambda nuclease; T7 nuclease; Exonuclease III; and T4 polymerase.

6. A method of generating recombinant DNA by homologous recombination without the use of ligases, comprising:
a) amplifying one or more target DNA molecules using an incomplete polymerase chain reaction procedure with a forward primer and a reverse primer, wherein
i) said forward primer terminates at its 5′ end in sequence A, wherein sequence A is 15-100 nucleotides in length;
ii) said reverse primer terminates at its 3′ end in sequence B, wherein sequence is 15-100 nucleotides long;
and wherein said incomplete polymerase chain procedure is characterized by a final step in which double stranded DNA is denatured and reannealed but not extended with the Taq DNA polymerase;
b) annealing DNA fragments produced in step b) with a linearized vector, wherein one end of said vector terminates in a single stranded region 15-100 nucleotides in length and having a sequence C that is exactly complementary to sequence A, and the other end of said vector terminates in a single stranded region 15-100 nucleotides in length and having a sequence D that is exactly complementary to sequence B;
d) transforming a host cell with the annealed complexes formed in step c).

7. The method of claim 6, wherein said host cell is a bacterium.

8. The method of claim 7, wherein said bacterium is of the species E. coli.

9. The method of claim 6, wherein the annealing of step c) is carried out in the presence of recA.

10. A method of cloning multiple DNA molecules, comprising:
a) combining 2-10 double stranded DNA fragments, each 40-5000 nucleotides long and each terminating on one end in a single stranded segment, either A or A′, 15-100 nucleotides long ending in a 5′ terminal phosphate and, on the other end, by a single stranded segment, B or B′, 15-100 nucleotides long ending in a 5′ hydroxyl, and wherein each A segment, consists of sequence that is exactly complementary to at least one B sequence;
b) subsequently or concurrently annealing the DNA fragments produced in step a) with a linearized vector, wherein one end of said vector terminates in a single stranded region having at one end a sequence C that is exactly complementary to sequence A′, and, at the other end, a single stranded region having a sequence D that is exactly complementary to sequence B′;
c) transforming a host cell with the annealed complexes formed in step b).

11. The method of claim 10, wherein each A and A′ segment has a sequence that is unique with respect to one another.

12. The method of claim 10, wherein the annealing of DNA fragments to one another and/or to vector is carried out in the presence of RecA.

13. The method of claim 10, wherein said host cell is a bacterium.

14. The method of claim 13, wherein said bacterium is of the species E. coli.

15. The method of claim 1, wherein the single stranded regions of said DNA fragments are generated by digestion of said DNA fragments using an exonuclease selected from the group consisting of: lambda nuclease; T7 nuclease; Exonuclease III; and T4 polymerase.

16. A kit comprising:
a) at least one oligonucleotide, wherein said oligonucleotide terminates at one end in sequence A, wherein sequence A is 15-100 nucleotides in length;
b) a vector that is, or can be, linearized to contain an end sequence that is exactly complementary to sequence A; and
wherein said kit does not include a DNA ligase.

17. The kit of claim 16, further comprising at least a second oligonucleotide, wherein said second oligonucleotide terminates at one end in sequence B, wherein sequence B is 15-100 nucleotides in length and wherein said vector, in addition to terminating at one end in a sequence exactly complementary to sequence A, terminates at the other end in a sequence that is exactly complementary to sequence B.

18. The kit of claim 17, further comprising RecA.

19. The kit of claim 17, further comprising a nuclease.

20. The kit of claim 19, wherein said nuclease is selected from the group consisting of: lambda nuclease; T7 nuclease; Exonuclease III; and T4 polymerase.
